(19) United States
(12) Patent Application Publication
(10) Pub. No.: US 2014/0277020 A1
(43) Pub. Date: Sep. 18, 2014

(54) QUADRICEPS TENDON STRIPPER/CUTTER

(71) Applicant: Arthrex, Inc., Naples, FL (US)

(72) Inventors: David C. Koogle, Bonita Springs, FL (US); Zachary A. Ingwer, Boca Raton, FL (US); Jacob A. Jolly, Naples, FL (US)

(73) Assignee: Arthrex, Inc., Naples, FL (US)

(21) Appl. No.: 14/204,760

(22) Filed: Mar. 11, 2014

Related U.S. Application Data
(60) Provisional application No. 61/777,354, filed on Mar. 12, 2013.

(51) Int. Cl. A61B 17/00; A61B 17/16; A61B 17/32

(52) U.S. Cl. CPC A61B 17/00008; A61B 17/320016; A61B 17/1635
USPC 606/167

(57) ABSTRACT

Instruments and methods that allow for a minimally invasive way to harvest a tissue graft which has been typically done as a full open procedure. The instrument is a hybrid stripper/cutter device provided with both stripping and cutting blades/mechanisms that allow stripping and cutting of the tissue graft with the same hybrid instrument. The present invention also provides a minimally invasive technique for stripping and cutting (resecting) a tissue graft by employing a single, hybrid stripper/cutter instrument. The technique provides a consistently good-quality graft with improved cosmetic results, and is simple and easily reproducible.

### CROSS-REFERENCE TO RELATED APPLICATIONS

This application claims the benefit of U.S. Provisional Application No. 61/777,354 filed Mar. 12, 2013, the disclosure of which is incorporated by reference in its entirety herein.

### FIELD OF THE INVENTION

The present invention relates to the field of surgery and, more particularly, to instruments and methods of harvesting quadriceps tendons.

### BACKGROUND OF THE INVENTION

The quadriceps tendon (QT or quad tendon) is becoming a popular graft for ligament surgery such as anterior cruciate ligament (ACL) revisions/reconstructions. The quadriceps tendon is larger and stronger than the patellar tendon or the semitendinosus tendon, and also stiffer and less prone to stretching or deformation. An ACL repaired with graft(s) from the quadriceps tendon results in almost no anterior knee pain postoperatively and a quick recovery.

Graft harvesting from the quadriceps tendon is difficult, however, as the existing surgical instruments require large incisions up the longitudinal axis of the femur to cut down to the level of the tendon, resulting in large postoperative scars. The quadriceps tendon is also unusually difficult to resect (cut) and, thus, dissecting a quadriceps tendon and resecting it to a desired length (amputating the end of the graft) is often challenging.

There is a need for instruments and techniques of harvesting a graft (for example, a quadriceps tendon or similar tissue grafts or tendons/ligaments) with minimal error and by simplified methods. An instrument designed to both strip and cut a tissue graft is also needed. Also needed are minimally invasive techniques for stripping/cutting a graft that are safe, provide a consistently good-quality graft with high cosmetic results, and are simple and easily reproducible.

### SUMMARY OF THE INVENTION

The present invention provides instruments and methods that allow for a minimally invasive way to harvest a tissue graft which has been typically done as a full open procedure. The device of the present invention is a hybrid stripper/cutter instrument provided with both stripping and cutting means that allow stripping and cutting of the tissue graft with the same hybrid instrument.

The present invention also provides a minimally invasive technique for stripping and cutting (resecting) a tissue graft by employing a single, hybrid stripper/cutter instrument. The technique provides a consistently good-quality graft with improved cosmetic results, and is simple and easily reproducible, requiring only a single incision.

These and other features and advantages will become apparent from the following description that is provided in connection with the accompanying drawings and illustrated embodiments.

### BRIEF DESCRIPTION OF THE DRAWINGS

FIG. 1 illustrates a perspective view of a hybrid stripper/cutter instrument according to an exemplary embodiment of the present invention.

FIG. 1(a) illustrates an expanded view of the stripper and cutter mechanisms of the hybrid stripper/cutter instrument of FIG. 1.

FIG. 1(b) illustrates an enlarged assembled view of the stripper and cutter mechanisms of the hybrid stripper/cutter instrument of FIG. 1(a).

FIG. 1(c) illustrates an enlarged view of the proximal end of the outer tube of the hybrid stripper/cutter instrument of FIG. 1.

FIG. 2 illustrates a schematic view of another exemplary hybrid stripper/cutter instrument of the present invention.

FIG. 2(a) is a left side view of the most distal end of the hybrid stripper/cutter instrument of FIG. 2.

FIG. 2(b) is a right side view of the most distal end of the hybrid stripper/cutter instrument of FIG. 2.

FIG. 3 illustrates a simplified perspective view of the hybrid stripper/cutter instrument of FIG. 2.

FIGS. 4-6 illustrate additional views of the hybrid stripper/cutter instrument of FIG. 1.

FIGS. 7-14 illustrate subsequent steps of a method of soft tissue (graft or tendon) harvesting with the hybrid stripper/cutter instrument of FIG. 1.

FIG. 15 illustrates a schematic representation of the harvested soft tissue of FIG. 14 secured within the femur and tibia.

### DETAILED DESCRIPTION OF THE PREFERRED EMBODIMENTS

The present invention provides surgical systems and methods for graft harvesting and replacement of an injured tendon/ligament or similar structure with such harvested graft. A hybrid combined stripper/cutter (combined graft stripping/cutting instrument) is provided with dual means for stripping and cutting soft tissue (tissue graft).

In an exemplary-only embodiment, the tissue graft is the quadriceps tendon; however, the instrument of the present invention can be employed for harvesting (stripping and cutting/resecting) any soft tissue or tissue graft or similar structure that is dimensioned/sized and cut for further use as a replacement of an injured tendon/ligament. For example, the combined stripper/cutter instrument of the present invention may be employed to harvest autografts such as patellar, hamstrings, Achilles and/or tibialis anterior/posterior. In exemplary embodiments only, the combined stripper/cutter instrument of the present invention may be employed to harvest central third bone-patellar tendon-bone (BPTB), semitendonosus and gracilis hamstring tendons, and central third quadriceps tendon, among others.

The present invention also provides methods of harvesting soft tissue (a tissue graft) by the steps of: (i) providing a hybrid (combined) stripper/cutter instrument; and (ii) employing the hybrid (combined) stripper/cutter instrument to both strip and cut/resect the tissue graft to a desired length.

Referring now to the drawings, where like elements are designated by like reference numerals, FIGS. 1-6 illustrate an exemplary hybrid stripper/cutter instrument 100, 100a of the present invention. FIGS. 7-15 illustrate methods of soft tissue harvesting (graft harvesting) with the hybrid instrument 100, 100a, and fixation of the harvested soft tissue in an exemplary method of ACL reconstruction.

Exemplary construct 100 of FIG. 1 is a hybrid stripper/cutting instrument (hybrid stripper/cutter) provided with a shaft 20 having a proximal end 21 and a distal end 22, a handle 10 provided at the proximal end 21, and a hybrid cutter/stripper device 50 provided at the distal end 22.

Hybrid cutter/stripper device 50 is formed of a tendon cutter 30 (first blade or edge 30, or tendon cutting mechanism 30) and a tendon stripper 40 (second blade or edge 40 or tendon stripper 40), both provided at the distal end 22 of the shaft of the instrument. In an exemplary embodiment, the tendon stripper 40 is an external mechanism, and the tendon cutter 30 is an internal mechanism contained within a tendon cutting housing 36 (which may have an exemplary “cigar cutter” configuration).

Tendon cutter 30 may be in the form of a blade or sharp edge that is designed to be housed within shaft 20 and/or a housing (such as housing 36) and then actuated by an actuator mechanism as detailed below. In an exemplary embodiment, the tendon cutter consists essentially of a tendon cutting blade (edge) that is designed to resect the tendon at the desired length. In additional embodiments, the tendon cutter consists of a tendon cutting blade and a housing for accommodating such blade when in the non-cutting position. The housing may be removably attached to the outer tube/shaft 20 or may be provided integral with it, and as desired. The tendon cutting blade may be replaceable and/or may be configured to be snapped onto the shaft 20 (or a connection element thereof such as a housing).

Tendon stripper 40 strips the graft (i.e., pre-sizes the graft to the desired width and depth) and the tendon cutter 30 then cuts the graft to the desired length, both operations/steps being conducted with a single incision and subcutaneously. Cutter housing 36 is provided with an opening 35, for example, a circular opening 35 with a diameter of about 11 mm to allow pre-sizing of grafts up to about 10 mm wide.

Shaft or outer tube 20 is also provided with a plurality of marks 26, for example, laser lines or laser marks 26 that indicate the graft length, as detailed below.

Handle 10 is provided with arms 13, 14 which may have different configurations and different actuating and arm locking mechanisms. The handle is designed to lock and unlock (as detailed below) to prevent the premature graft cutting during the stripping operation. The handle is pushed down to swing the arm lock 12 (locking mechanism 12) out of the way, to squeeze the two arms of the handle and cut the graft at the desired length. The handle and locking mechanism may have additional designs, for example, a spring 11 of FIGS. 1(a)-1(c) or flat form spring 11a of FIGS. 1 and 2(a). Additional views of how the two arms squeeze together to lock the instrument are shown in FIGS. 4-6.

At least one of the tendon stripper blade 40 and the tendon cutting blade 30 may be integrally or removably attached to the outer tube/shaft 20. The tendon stripper blade 40 and the tendon cutting blade 30 may be interchangeable with each other, may be removable, may be sterilizable (autoclavable), and may be provided as single-use only devices (disposable) or as multiple-use devices. In particular applications, the tendon stripper blade 40 and the tendon cutting blade 30 may be designed so that one may be snapped onto the instrument while the other may be snapped out of the instrument (or vice-versa). The tendon stripper blade 40 and the tendon cutting blade 30 may be also designed so that one can be removed from the instrument, while the other one can remain securely attached to the instrument.

In use, tendon stripper 40 separates surrounding tissue from graft or tendon 80, as detailed below, and between a first location and a second location indicated by the distance traveled by instrument 100 along a length of the stripped tendon. Tendon stripper 40 may be provided in the exemplary shape and configuration shown in FIG. 1(a), i.e., with arms/blades 41 spaced apart a distance “w” that indicate the width of the stripped graft/tendon that undergoes stripping of surrounding soft tissue. Tendon stripper 40 may have, however, various configurations, for example, may be provided as a semicircular or circular blade/edge, as long as soft tissue separates from the graft/tendon. In an exemplary embodiment, the tendon stripper 40 consists of a tendon stripping blade or edge. The tendon stripping blade may be replaceable. In other embodiment, the tendon stripping blade may be configured to be snapped onto the shaft 20 (or a connection element thereof).

In the exemplary embodiments of FIGS. 2 and 3, tendon cutter 30 of device 100a is designed to engage (mate with) a tendon cutting replaceable blade 30 having an opening 35 (for example, a circular opening 35 with a diameter of about 11 mm to allow pre-sizing of grafts up to about 10 mm wide) within the cutter housing 36. The tendon cutting blade is connected to a blade linkage arm 42. When the arms of handle 10 are squeezed, and as detailed below, an inner linkage arm 25 of an actuator mechanism (also shown in FIG. 1(c), for example) is advanced forward, causing the blade linkage arm 42 to travel up (i.e., in an upward direction) and to actuate the tendon cutting blade to shear the graft at the desired length. The blade may have a fan-shaped configuration, or a semicircular or circular configuration, or a cigar cutter-shape configuration, among many others.

Tendon stripper 40 (tendon stripping blade 40) also engages and mates with the outer tube 20. Various sized stripper blades can be attached to strip different width and depth tendon sizes and shape variations.

The present invention also provides a method of harvesting a graft with instrument 100, 100a of FIGS. 1-6. According to an exemplary-only embodiment, a method of harvesting (sizing and cutting) a tissue graft to desired dimensions (i.e., width and length) comprises the steps of: (i) providing a hybrid (combined) stripper/cutter instrument 100, 100a; and (ii) employing the hybrid (combined) stripper/cutter instrument 100, 100a to both strip and cut/resect a tissue graft.

According to another exemplary method of the present invention, and with reference to FIGS. 7-15, a graft is harvested (sized and cut to specific dimensions) and then secured within at least a bone tunnel or socket by the following steps:
1. A small incision 91 is made above the knee cap of knee 90;
2. After making the skin incision 91, a retractor 92 (FIG. 9) may be used to lift the skin while an arthroscope 93 is placed under the skin to visualize the course of the tendon 80. The arthroscope 93 (FIGS. 9 and 9(a)) is advanced proximally until the end of the tendon is visualized. Turn the arthroscope upwards so the light can be seen through the skin—this is the proximal limit of the graft harvest (tendon). Mark the skin at this point of reference M (FIG. 9(a)).
3. A desired blade 94 is placed onto a handle 94a and advanced to bulge the skin and reach marker spot M of the skin (FIGS. 10-10(b)).
4. A cutting instrument 95 (for example, a parallel knife blade) is employed to score the tendon 80 (i.e., to cut the tendon at the top) above the knee cap (FIGS. 11 and 12);
5. The tendon 80 is dissected from the patella and the distal end of the tendon 80 (exemplary quad tendon) is grasped; tension is applied to the graft, allowing about 3 cm to be exposed and prepared (FIG. 12);
6. At least one suture 70 is attached to the dissected tendon 80 to form a stitched tendon 88 (a tendon with a distal end stitched region); the distal end of the graft may be whipstitched for example using a #2 FiberLoop® with a curved or straight needle (FIG. 13);
7. Hybrid stripper/cutter instrument 100 is brought in the vicinity of the stitched tendon 88 and the suture 70 is passed with the attached stitched distal end 88 of the tendon through opening 35 (for example, an exemplary 11 mm diameter opening 35) of the housing 36 of the hybrid graft stripper/cutter instrument 100; the handle 10 must be locked to prevent premature cutting of the graft (FIG. 13);
8. With the suture 70 and stitched tendon 88 passed through opening 35, instrument 100 is pushed forward (in the direction of arrow A of FIG. 13), along the length of the tendon, to strip the tendon to the desired length (indicated by laser lines 26 on outer tube 20) with blade 40, as shown in FIGS. 13(a)-13(c); the hybrid stripper/cutter instrument 100 is advanced proximally and rotation may facilitate advancement to separate adjacent surrounding soft tissue from the graft; and
9. Once the stripper/cutter instrument 100 reaches the desired depth (as indicated by the skin marking M and instrument measurements and as shown in FIG. 13(b)), the handle is unlocked (FIG. 13(c)) and the trigger is squeezed to cut the proximal tendon (graft), i.e., the tendon cutting blade or cutter 30 is activated (by squeezing the grip handle 10, for example) to advance the inner rod (inner linkage arm 25) to travel up and cause the cutting blade to cut the tendon (cut the bottom of the tendon) and to release the entire cut-off tendon. The resected/cut-off tendon 180 (FIG. 14) has a length of about 7 to 8 cm and a width of about 7 to 11 mm.

FIG. 15 illustrates a schematic representation of knee 90 undergoing ACL reconstruction employing the graft construct 180 (tissue construct 180) formed according to an embodiment of the present invention. Resected/cut-off tendon 180 (FIG. 14) obtained by a method and with the instruments of the present invention is secured within exemplary femoral and tibial tunnels (sockets) 90a1, 90b1, each formed in femur 90a and tibia 90b, respectively, to obtain final repair 200.

Tendon 180 may be stitched/reinforced at both ends with a flexible suture loop/needle construct similar to the FiberLoop® construct detailed and disclosed in U.S. Pat. No. 8,298,284 issued on Oct. 30, 2012, the disclosure of which is incorporated by reference herein in its entirety. As detailed in U.S. Pat. No. 8,298,284, the suture loop/needle construct includes a continuous loop of suture (preferably a continuous loop of #2 FiberWire® suture (U.S. Pat. No. 6,716,234), sold by Arthrex, Inc. of Naples, Fla.) with both ends attached to a free floating thin needle (such as a thin nitinol needle). The thin needle is preferably straight and easy to handle, without instruments. The needle moves freely on the suture loop to recenter itself after passing through tissue and to facilitate even tensioning. The free floating needle is not swaged on the suture loop so, after passing the suture loop construct through the tissue to be treated, the needle may be recentered if the suture strands are uneven.

The tendon may be reinforced by employing a reinforcement material 60, for example a suture, tape, weave or mesh, among many others. In an exemplary only embodiment, the reinforcement material 60 is in the form of a wide “tape like” material provided on the resected tendon to form a loop that can be attached to a fixation device (for example, a RetroButton® (U.S. Patent Application Publication No. 2008/0046009), ACL Tightrope® (U.S. Pat. No. 8,460,379) or other suture loop/button construct). The wide “tape like” area is placed over the tendon or ligament and permits the surgeon to include the construct into the tendon/ligament stitches. The “tape like” material may include suture, Fiberwire® (U.S. Pat. No. 6,716,234), FiberTape® (U.S. Pat. No. 7,892,256), absorbable suture, tape, weave, or mesh, or any other material or combination of such materials. The “tape like” material 60 is provided on the ligament or tendon, along a length thereon, and whipstitched to provide additional fixation when the ligament or tendon is implanted. The material may be absorbable or non-absorbable. Preferably, the length of the construct approximates the length of the tendon or ligament that would normally be stitched.

Tendon 180 may be attached to any fixation device (for example, a RetroButton® (U.S. Patent Application Publication No. 2008/0046009), ACL Tightrope® (U.S. Pat. No. 8,460,379) or other suture loop/button construct) and/or any bone tunnel.

The femoral and tibial tunnels (sockets) 90a1, 90b1 may be formed by a known technique, or alternatively, by using a retrodrill technique. According to the retrodrill technique, the tibial or femoral sockets are prepared by employing a retrodrill device provided with a retrodrill cutter detachable from a retrodrill guide pin, in the manner described in U.S. Pat. No. 7,238,189, issued Jul. 3, 2007, entitled “ACL Reconstruction Technique Using Retrodrill,” the entire disclosure of which is incorporated by reference herein in its entirety.

Preferably, the step of securing the graft 180 in at least the tibial and femoral sockets or tunnels may be accomplished by plugging the target tunnel with a synthetic plug, bone plug or screw to contain and secure the graft 180 in the tunnel or, alternatively, by employing knotless lateral fixation using, for example, two knotless fixation devices (such as interference screws or graftbolt-type fixation devices). The fixation devices may be preferably formed of a translucent or transparent polymer material, preferably bioabsorbable materials such as polyglycolic or polylactic acid polymers.

According to yet another embodiment, graft insertion and fixation may be conducted by employing a continuous loop/button construct provided with a button, preferably of titanium alloy, and a continuous loop attached to the button. The button has an oblong configuration and a width that is preferably less than about 1 mm narrower than the width of the drill hole through which the button is inserted and subsequently passed through. The button is provided with an inside eyelet that allows the passage of the continuous loop. In an exemplary embodiment, the suture loop may be a single high strength suture such as FiberWire® suture. In another exemplary embodiment, the continuous loop may be formed of a plurality of suture strands configured to separate from a single strand to a plurality of strands in a continuous loop. In yet another embodiment, the loop is an adjustable loop (forming a TightRope® ACL construct 150 as shown in FIG. 14) which consists of two interconnected, adjustable flexible loops formed by splicing a suture strand in a manner disclosed in U.S. Pat. No. 8,439,976 issued on May 14, 2013 and U.S. Pat. No. 8,460,379 issued on Jun. 11, 2013, the disclosures of both of which are incorporated by reference in their entireties herein.

When employing an adjustable button/loop construct 150 such as the suture loop/button construct disclosed in U.S. Pat. Nos. 8,439,976 and 8,460,379 (comprising an adjustable loop formed of two interconnected loops (two eyesplice loops) and two splices), the resected tendon 180 may be attached directly or indirectly to the adjustable button/loop construct using various attachment techniques.

The present invention also provides a minimally invasive technique for stripping and cutting (resecting) a tissue graft by employing hybrid stripper/cutter instrument 100, 100a. The technique provides a consistently good-quality graft with improved cosmetic results, and is simple and easily reproducible. After the tendon has been cut with a cutting device (such as a knife, for example, a parallel knife blade), the device 100, 100a can be inserted percutaneously with the stitched tendon passed through the tip opening 35. The device 100, 100a is pushed forward stripping the tendon to the desired length with stripping blade 40 (the length being indicated by the laser marks on the outer surface of tube 20) and then the tendon is cut with cutting mechanism 30 to the desired length, completing the harvest. The device 100 provides (and accomplishes) both tendon stripping and cutting.

The harvested graft(s) may be further employed in various ligament/tendon/graft reconstructions known in the art, for example, ACL reconstructions among many others.

The minimally invasive quad tendon harvest system of the present invention provides the following benefits:

Quad Tendon Graft Cutting Guide
1. Low profile blade facilitates subcutaneous tendon cutting at fixed widths of 9, 10 and 11 mm;
2. Depth limiter ensures proper graft thickness;
3. Length measurements on shaft allow visual indication of graft cutting length.

Quad Tendon Stripper-Cutter
1. Sharp leading edge strips graft away from surrounding tissue;
2. Cutter allows subcutaneous resection of proximal attachments without a second incision;
3. Depth markings on shaft allow visual confirmation of graft length.

While the present embodiments are described herein with reference to illustrative figures for particular applications, it should be understood that the embodiments are not limited thereto. Those having ordinary skill in the art and access to the teachings provided herein, will recognize additional modifications, applications, embodiments and substitution of equivalents all falling within the scope of the presented embodiments.

What is claimed is:

1. A method of harvesting a graft, comprising the steps of
releasing a distal end of a graft while a proximal end of the graft remains attached to a proximal attachment point;
placing the distal end of the graft through an opening of a stripper/cutter instrument; and
advancing the stripper/cutter instrument along a length of the graft and towards the proximal end of the graft, and then cutting the graft subcutaneously at a desired length with the stripper/cutter instrument.

2. The method of claim 1, wherein the steps of releasing the distal end of the graft and cutting the graft are conducted through a single incision.

3. The method of claim 1, further comprising the step of stitching the distal end of the graft prior to the step of placing the distal end of the graft through the opening of the hybrid stripper/cutter instrument.

4. The method of claim 1, wherein the stripper/cutter instrument includes a first edge or blade that strips the graft away from surrounding soft tissue, and a second edge or blade that cuts the graft at the desired length.

5. The method of claim 4, wherein the first edge is integral with the second edge.

6. The method of claim 4, wherein at least one of the first and second edges is replaceable.

7. The method of claim 1, wherein the opening has a width of about 10 to 11 mm.

8. A method of stripping and cutting a tendon with a single instrument, the method comprising the steps of:
exposing a tendon;
aligning the exposed tendon with a combined tendon stripper/cutter instrument so that at least a portion of the tendon extends through a most distal opening of the combined tendon stripper/cutter instrument; and
advancing the combined tendon stripper/cutter instrument down a length of the tendon, from a first location to a second location on the tendon.

9. The method of claim 8, further comprising the step of actuating a cutting blade of the combined tendon stripper/cutter instrument to resect the tendon at the second location and to obtain a resected tendon.

10. The method of claim 9, wherein the resected tendon has a width of about 7 to 11 mm and a length of about 7 to 8 cm.

11. The method of claim 8, wherein the step of advancing the combined tendon stripper/cutter instrument down a length of the tendon further comprises advancing the tendon through the most distal opening to allow stripping of soft tissue surrounding the tendon.

12. A hybrid stripper/cutter instrument for graft striping and cutting, comprising:
a handle;
an outer tube attached to the handle, the outer tube having a proximal end and a distal end;
a hybrid stripper/cutter provided at the distal end of the outer tube;
an actuator mechanism for actuating a cutting blade of the hybrid stripper/cutter; and
a locking mechanism for locking the hybrid stripper/cutter at one or more different locking positions.

13. The hybrid stripper/cutter instrument of claim 12, wherein the actuator mechanism comprises a rack and pinion assembly.

14. The hybrid stripper/cutter instrument of claim 13, wherein the locking mechanism comprises a slot in a pinion gear of the rack and pinion assembly, the slot having a plurality of detents; a pin that is configured to sit in one of the plurality of detents when the pinion gear is locked; and a return spring.

15. The hybrid stripper/cutter instrument of claim 12, wherein the hybrid stripper/cutter instrument comprises a cutting blade housed in a housing attached to the outer tube, and a stripping blade attached to the outer tube.

16. The hybrid stripper/cutter instrument of claim 15, wherein at least one of the cutting blade and the stripping blade is interchangeable and disposable.

17. The hybrid stripper/cutter instrument of claim 15, wherein at least one of the cutting blade and the stripping blade is replaceable.

18. The hybrid stripper/cutter instrument of claim 15, wherein at least one of the cutting blade and the stripping blade is sterilizable.

19. The hybrid stripper/cutter instrument of claim 15, wherein one of the cutting blade and the stripping blade is removed from the instrument, while the other of the cutting blade and the stripping blade remains securely attached to the instrument.

20. The hybrid stripper/cutter instrument of claim 12, wherein the outer tube is provided with a plurality of laser marks to indicate a length of the stripped and cut graft.
